(19) United States
(12) Patent Application Publication
(10) Pub. No.: US 2006/0089581 A1
(43) Pub. Date: Apr. 27, 2006

(54) Knee orthosis

(76) Inventor: Gerhard Lambert, Salzburg (AT)

Correspondence Address: Fitch, Even, Tabin & Flannery, Suite 1600, 120 South LaSalle Street, Chicago, IL, 60603-3406, US

(21) Appl. No.: 11/236,341

(22) Filed: Sep. 27, 2005

(30) Foreign Application Priority Data
Sep. 27, 2004 (DE) 10 2004 046 743.9

(51) Int. Cl. A61F 5/00

(52) U.S. Cl. 602/16; 602/26

(57) ABSTRACT

Knee orthosis having a femoral orthosis member and a tibial orthosis member, which are joined to one another by way of lateral joints, the bending of the joints being defined in each case by two guideways, especially guide slots, on the one hand and by associated cams, especially pin followers, on the other hand, the guideways, especially guide slots, extending along two fixed centrodes, especially fixed centrode circles, that intersect at a point.

### FIELD OF THE INVENTION

The invention relates to a knee orthosis having a femoral orthosis member and a tibial orthosis member in accordance with the present application.

### BACKGROUND OF THE INVENTION

Knee orthoses of the mentioned kind are used therapeutically and post-operatively for supporting and securing the joint system at the knee. The orthosis, which for this purpose is affixed by means of at least one cuff both to the thigh and to the lower leg, has a joint system that is intended to imitate an anatomically correct movement sequence of the knee joint. It is important here that the knee joint, which has usually been weakened or damaged after injury or an operation, does not become mechanically stressed by incongruities between the functional anatomy of the human knee joint and the technological joint guidance provided by the knee orthosis. If the guidance of the orthotic joint is not anatomically correct, such incongruities can result in shear, compressive and/or tensile stresses being exerted on the human knee joint, which, especially when the orthosis is securely anchored to the leg, in the worst case results in further damage to the knee joint, which must be avoided at all costs.

An anatomically and functionally correct imitation of the human flexion sequence at the knee is therefore extremely difficult, because the circumference of the femoral condyle is twice as long as the tibial articular surface, so that a pure rolling movement of the joint is not possible. Investigations into the real movement sequences between the femoral condyle and tibial condyle show that the femoral condyle rolls and glides on the tibia, so that posterior luxation is prevented. During flexion, stress is specifically exerted on the anterior cruciate ligament, which pulls the femoral condyle anteriorly or maintains it in an anterior position. In other words, during flexion the anterior cruciate ligament is responsible for the forward gliding movement of the femoral condyle, while during extension the posterior cruciate ligament acts in an analogous manner posteriorly.

In order to imitate that extremely complex movement sequence there are used for knee joints polycentric joints by means of which it is possible to avoid the above-mentioned incongruities between the functional anatomy of the human knee joint and the technological joint guidance of the knee orthosis. Such polycentric joints do not have a rigidly defined pivot point but have a pivot point that constantly changes its position in the course of flexion or extension of the leg. Accordingly, the instantaneous pivot point of the knee joint, and of the orthotic joint imitating the flexion of the knee, is dependent in respect of its position in two-dimensional or three-dimensional space upon the flexion angle of the joint, the large number of respective pivot points describing a curve which is referred to as a centrode.

A realisation of such a polycentric joint system is described, for example, in EP 0 361 405 B1 and the corresponding DE 689 28 511 T2, which relates to a knee orthosis having controlled multiaxis movement. The joint mechanism therein, which is shown in FIG. 9, comprises two cam slots 13a, 13b and two cam pin followers A, B, which are affixed to mutually associated joint members 13, 11, one cam slot 13a being arranged in a transverse plane and serving to facilitate the forward movement of the upper joint member 11, while the second cam slot 13b is arranged in a longitudinal orientation and provides a long arcuate segment for a unicentric phase of the joint arthrokinematic system. At the beginning of flexion, in the joint mechanism therein the upper cam pin follower B moves forward and downwards in the curved slot 13b which extends in the longitudinal direction. In the course of that movement, the upper cam pin follower B approaches the lower cam slot 13a arranged in the horizontal plane, the cam pin follower A guided in that lower cam slot 13a being displaced towards the rear.

A disadvantage of the structure therein is the fact that during flexion of the femur-side and tibia-side orthosis members 11, 13, the two cam pin followers A, B move simultaneously in the associated cam slots 13a, 13b, the movements of the cam pin followers A, B taking place in different directions which are determined by the cam slots 13a, 13b. The paths of the cam pin followers A, B in the cam slots 13a, 13b can be adhered to exactly only by the cam pin followers A, B sliding laterally along the boundaries of the cam slots 13a, 13b, the cam pin followers A, B rubbing against the edges of the cam slots 13a, 13b, which impedes movement and results in associated wear to the cam pin followers A, B and to the edges. Furthermore, it can be seen clearly from FIG. 9 that in the event of flexion starting from the extended position of the leg, the lower cam pin follower denoted by reference letter A, which runs in the horizontal transverse plane, is first displaced into the lower right-hand corner (as shown in the drawing) of the horizontal cam slot 13a, with great pressure being exerted on the lower edge of the cam slot 13a so that there is a risk that flexion will be inhibited. Such problems intensify with increasing use of the knee orthosis described therein, because the continued friction will result in greater wear. Accordingly, the functional ability of the knee orthosis described in EP 0 361 405 B1 is not lastingly ensured, it being necessary to replace the joint mechanism therein relatively frequently.

### SUMMARY OF THE INVENTION

The problem underlying the invention is to provide a knee orthosis having polycentric joint guidance, by means of which an anatomically correct flexion sequence is ensured and the above-mentioned problems are avoided and which is at the same time cost-effective to manufacture.

That problem is solved by a knee orthosis according to the features described hereinafter.

The problem is solved, in particular, by a knee orthosis having a femoral orthosis member and a tibial orthosis member, which are joined to one another by way of lateral joints, the bending of the joints being defined in each case by two guideways, especially guide slots, on the one hand and by associated cams, especially pin followers, on the other hand, which guideways, especially guide slots, extends along two fixed centrodes, especially fixed centrode circles, that intersect at a point.

An important aspect of the invention is that the guide slots are formed as fixed centrode circles having a uniform radius, along which the pin follower associated with the respective fixed centrode circle moves.

In accordance with an embodiment, the two guideways, especially guide slots, each extends as far as the intersection point of the associated centrodes, forming a continuous triangular cam guide means. Furthermore, the ends of the two guideways, especially guide slots, remote from the intersection point of the centrodes, and the intersection point of the centrodes are located at the corners of a notional equilateral triangle, the distance between the cams, especially pin followers, associated with the respective guideways corresponding to the side length of the notional equilateral triangle.

In this extremely simple way it is ensured that on flexion of the orthotic joint only one pin follower is moved along a concentric circular path, namely the portion of a fixed centrode circle associated with the guideway, the respective other pin follower forming the respective pivot point. Accordingly, the pin followers are reliably prevented from inhibiting one another.

Because the movement sequence of the pin follower moving in the guide slot is exactly defined by its linkage to the orthosis member on the one hand and to the other pin follower acting as pivot point on the other hand, it is not absolutely necessary for the moving pin follower to make contact with the edges of the guide slot, so that wear to the pin follower and to the edges is avoided.

In the course of flexion of the orthotic joint, one pin follower acts as pivot point for the movement of the other pin follower along the guide slot until the moving pin follower, starting from the intersection point of the two guide slots, reaches the end of the guide slot associated with the pin follower, whereupon the other pin follower, which previously acted as pivot point, moves from the end of its associated guide slot as far as the intersection point of the centrodes.

Because the two guide slots are formed by segments of fixed centrode circles each having an identical radius, the respective centre point of which is defined by an end region of the opposite guide slot, a movement sequence that is harmonious and almost frictionless in respect of the pin followers and guide slots is possible and provided, which movement sequence corresponds to an anatomically correct flexion sequence of the human knee.

Since in every phase of flexion only one pin follower is moved in its associated guide slot, according to the invention there is no possibility of obstruction, sticking or tilting as in the prior art shown in FIG. 9.

In accordance with an embodiment of the invention, the intersection point of the centrodes associated with the two guideways is located anteriorly, the angle-bisecting line passing through the intersection point of the centrodes located along the notional equilateral triangle being inclined downwards in the direction from posterior to anterior between the two guideways. In that way, an upright standing position as well as overextension of the leg up to a hyperextension of up to −10° are facilitated.

Furthermore, the angle-bisecting line encloses with the longitudinal axis of the extended knee orthosis on the anterior femoral side an angle (α) of from 100° to 120°, especially an angle of approximately 110°.

According to the invention, the two guideways of the pin followers extend over an arc angle β of up to 60°, so that a transition-free displacement of the pivot point is possible. That pivot point displacement takes place in advantageous manner when the knee joint is flexed half way between maximum extension and maximum flexion. Accordingly, the displacement of the pivot point takes place at a position in which the knee joint is subject to minimal load, which makes a substantial contribution to the especially smooth running of and lack of wear to the orthotic joint according to the invention.

As already mentioned above, the knee orthosis according to the invention allows hyperextension of up to −10°. This is achieved by so matching the positions of the two guideways, and of the cams associated therewith, to one another that the connecting line between the two cams, especially pin followers, in the extended state is inclined upwards in the direction from posterior to anterior. The inclination, which is expressed by an angle γ, is preferably 5°≦γ≦20°. In accordance with a preferred embodiment, the angle γ is approximately from 8° to 12°, especially 10°.

It should also be mentioned that the guideways, or the continuous cam guide means, form part of a first splint associated with the femoral or tibial orthosis member and the cams form part of a second splint associated with the respective other orthosis member. In other words, the cam guide means can be provided on the tibial orthosis member, as shown according to the invention in the drawings, while the cams are mounted on the femoral orthosis member. The knee orthosis also functions in accordance with the invention, however, when the cam guide means are mounted on the femoral orthosis member and the respective associated cams on the tibial orthosis member. Accordingly, the functionality of the knee orthosis according to the invention is independent of gravity and in that respect can also be used in recumbent patients or patients having their leg elevated, without the risk of the joint's becoming stuck or unintentionally locked.

Furthermore, the orthotic joint according to the invention is associated with posterior and/or anterior stop means for limiting bending and/or extension. According to an embodiment of the invention, the stop means consist of simple bores which are provided in the respective orthosis members and allow the passage of a limiting pin against which the respective outer edges of the femoral or tibial orthosis members rest. The bores are so provided that virtually all limit angles for bending and/or extension that are considered reasonable by an orthopaedic surgeon can be fixed, or flexion or extension of the knee joint is possible only within set limits, so that the knee orthosis can be adjusted individually to a wide range of support functions.

Otherwise, the joints are provided with at least one lateral outer covering which is especially equipped with a snap fastening, hook fastening or interlocking fastening. Press studs or a simple hook and loop fastening, for example, are suitable for that purpose.

Further embodiments of the invention will be found in the description hereinafter.

### BRIEF DESCRIPTION OF THE DRAWINGS

The invention is described below with the aid of an embodiment, which is explained in greater detail with reference to the Figures, wherein

FIG. 1 is a diagrammatic view of a tibial orthosis member;

FIG. 2 is a diagrammatic view of a femoral orthosis member;

FIG. 3-8 show diagrammatic views of a knee orthosis according to the invention having different flexion angles; and

FIG. 9 shows a knee orthotic joint according to the prior art.

### DETAILED DESCRIPTION OF THE PREFERRED EMBODIMENTS

In the description which follows, parts that are identical or have an identical action have been given the same reference numerals.

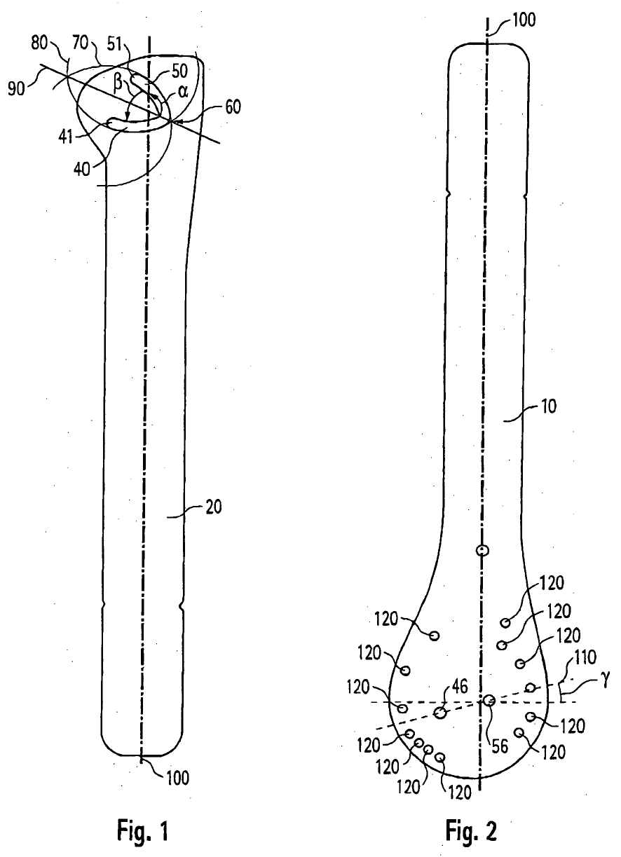

FIG. 1 shows a tibial orthosis member 20 having a longitudinal axis 100 and two guide slots 40 and 50. The guide slots 40 and 50 each extends over a portion of an associated fixed centrode circle 70, 80 and intersect at intersection point 60. On the side of the guide slots 40, 50 remote from the intersection point 60 there are located their respective ends 41, 51 which act as stop and pivot point for the cams, especially pin followers 45, 55, running in the guide slots 40, 50. An angle-bisecting line 90 passes through the intersection points of the fixed centrode circles 70, 80 and through the intersection point 60 of the guide slots 40, 50. The angle α of the angle-bisecting line 90 in respect of the longitudinal axis of the knee orthosis 100 is 110°. An arc angle β is shown in FIG. 1 between the two guideways, or guide slots 40, 50. That angle is 60° and in each case defines a side of an equilateral triangle.

FIG. 2 shows the femoral orthosis member 10 associated with the tibial orthosis member from FIG. 1, the longitudinal axis 100 of the knee orthosis being indicated. The femoral orthosis member has two bores 46, 56 for the attachment of cams or pin followers 45, 55 which are arranged along a cam-connecting line 110. The cam-connecting line 110 encloses an angle γ of about 15° with a horizontal line perpendicular to the longitudinal axis 100 of the knee orthosis. The femoral orthosis member 10 also has stop means 120 in the form of bores through which a limiting pin (not shown) can be passed and arrested therein. In this connection it should be mentioned that to form the knee orthosis there are provided in advantageous manner on each side of the orthosis two femoral orthosis members 10 between which the tibial orthosis member 20 is guided with the aid of the guide slots 40, 50 and the pin followers 45, 55, so that a limiting pin, introduced through one of the stop holes 120, after being guided past the tibial orthosis member is held in a further stop hole 120 of a second associated femoral orthosis member 10 and accordingly is anchored by each of its ends in a stop hole 120.

In advantageous manner, the knee orthosis has on each side a joint structure consisting of two femoral orthosis members 10 and one tibial orthosis member 20 but, if the knee orthosis is of suitably rigid construction, a joint guide means on one side can be sufficient for stabilising and guiding the knee joint.

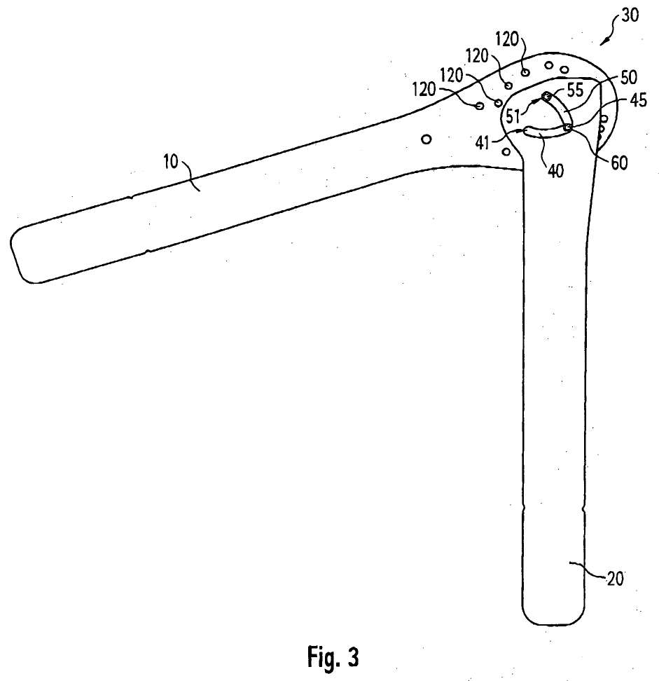

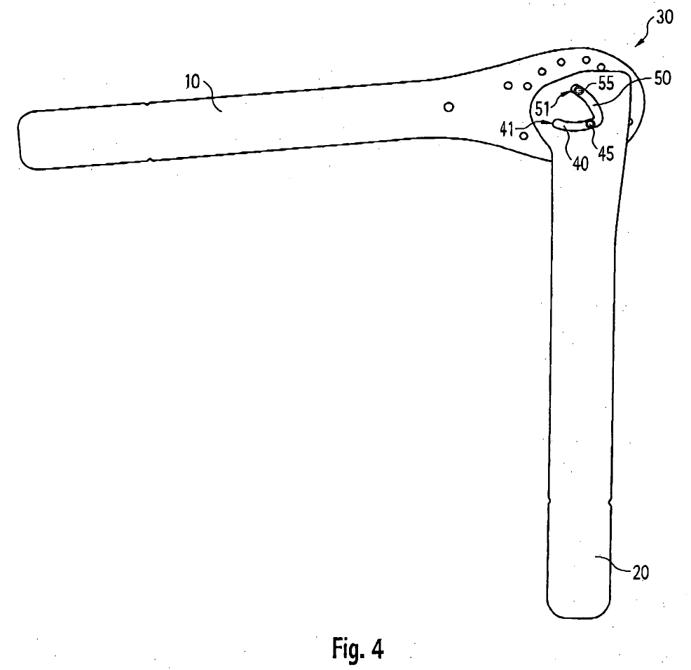

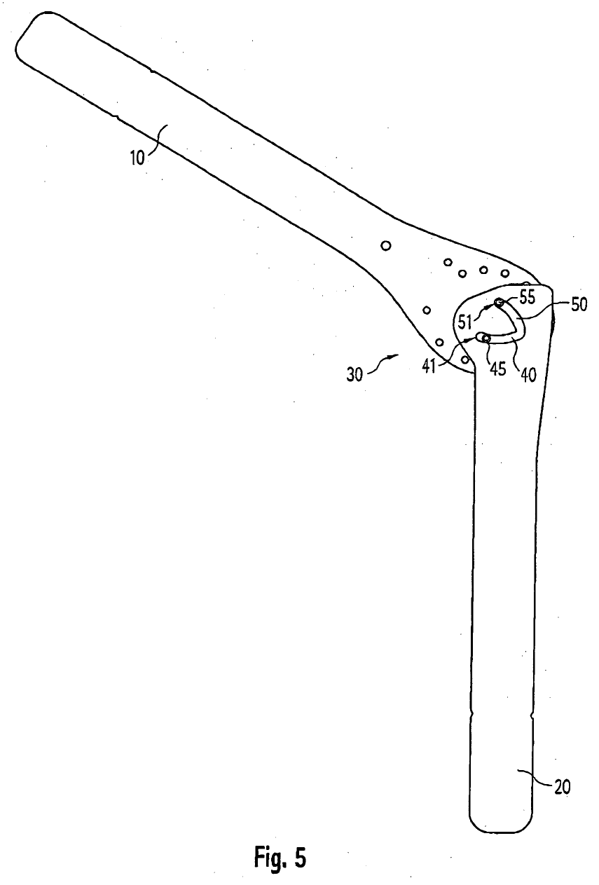

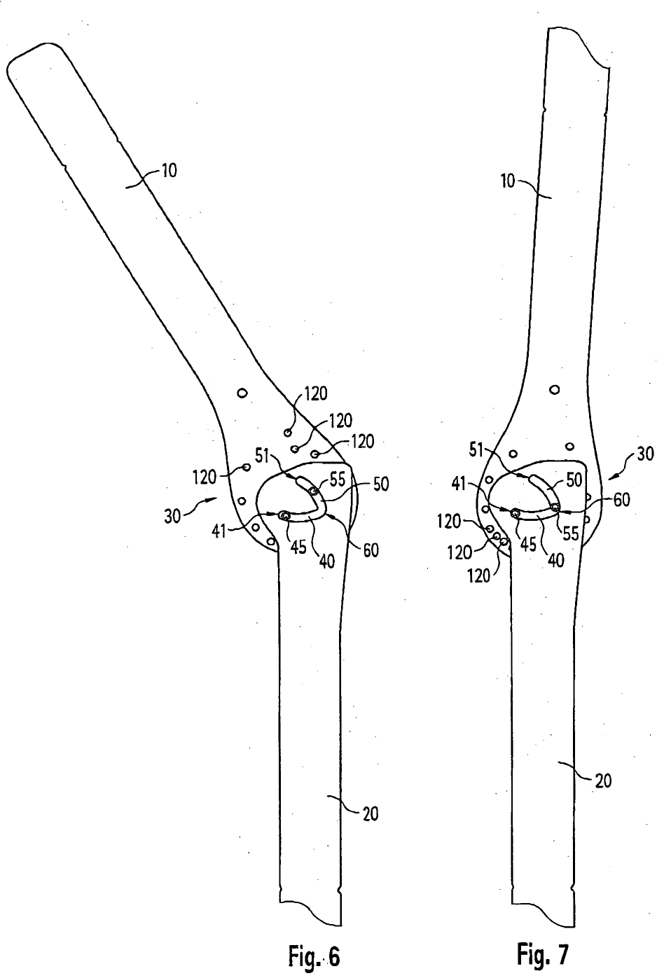

FIGS. 3 to 8 show a diagrammatic view of a knee orthotic joint 30 according to the invention in part section, there being mounted on a femoral orthosis member 10 two pin followers 45, 55 which run in guide slots 40, 50 provided on a tibial orthosis member. According to FIG. 3, the orthotic joint 30 is shown at its maximum flexion angle in which the pin follower 55 comes to rest at the end 51 of the guide slot 50, while the pin follower 45 is arranged at the intersection point 60 of the two guide slots 40, 50. As can be seen in FIGS. 4, 5 and 6, on extension of the orthotic joint 30 the pin follower 45 is displaced rearwards along the guide slot 40 in the direction of the end 41 of the guide slot 40, while the pin follower 55, which is arranged at the end 51 of the guide slot 50, acts as pivot point for the movement of the pin follower 45 along the portion of the fixed centrode circle 80 along which the guide slot 40 is formed.

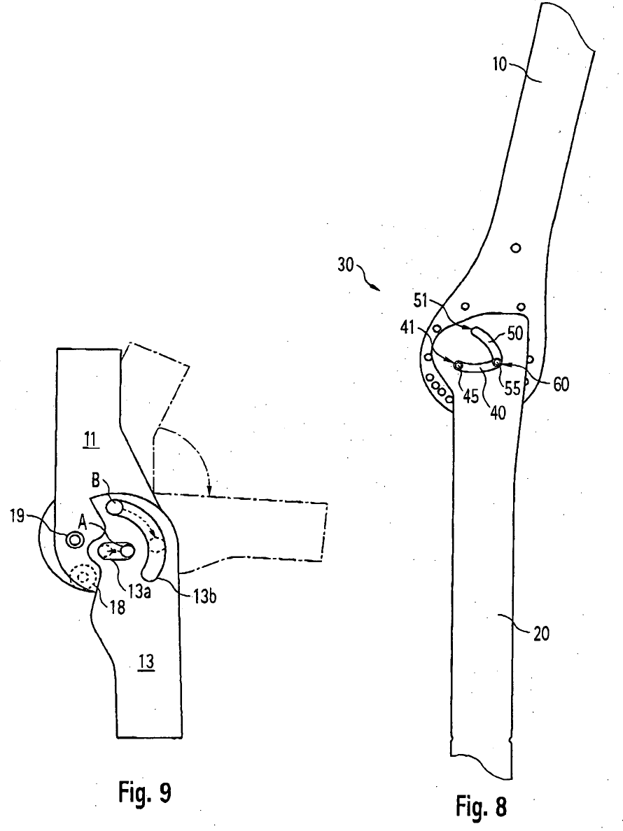

In the state where the knee orthosis is at half the maximum flexion angle, both pin followers 45 and 50 are arranged in the respective associated ends 41, 51 of the respective guide slots 40, 50. In the event of the orthotic joint 30 being extended further, the pin follower 45 arranged at the end 41 of the guide slot 40 acts as pivot point, the pin follower 55 moving along the fixed-centrode-circular guide slot 50 in the course of the further extension as far as the intersection point 60 of the guide slots 40, 50. Because, on the one hand, the angle-bisecting line 90 between the guide slots 40, 50 is inclined downwards and the connecting line 110 between the pin followers 45, 55 in the extended state extends upwards in the direction from posterior to anterior, hyperextension of up to −10° is possible. Such hyperextension is shown in FIG. 8.

It should be pointed out here that all the above-described components, alone and in any combination, especially the details shown in the drawings, are claimed as being important to the invention. Modifications thereof will be familiar to the person skilled in the art.

### REFERENCE NUMERALS

10 femoral orthosis member
20 tibial orthosis member
30 joint
40 guide slot
41 end of the guide slot
45 pin follower
46 bore for pin follower
50 guide slot
55 pin follower
56 bore for pin follower
60 intersection point
70 fixed centrode circle
80 fixed centrode circle
90 angle-bisecting line
100 longitudinal axis of knee orthosis
110 cam-connecting line
120 stop means

What is claimed is:

1. A knee orthosis comprising:
a femoral orthosis member,
a tibial orthosis member,
lateral joints joining said femoral orthosis member and said tibial orthosis member to one another,
guide means selected from the group consisting of two guideways, and two guide slot guideways, and
cam means associated with said guide means and selected-from the group consisting of cams, and pin follower cams, said guide means on the one hand and said cam means on the other hand being operatively arranged to define in each case the bending of said joints, wherein said guide means extends along paths selected from the group consisting of two fixed centrodes, and two fixed centrode circles, said paths intersecting at a point.

2. A knee orthosis according to claim 1, wherein said guide means each extends as far as the intersection point of the associated centrodes so as to form a continuous triangular cam guide means.

3. A knee orthosis according claim 1, wherein the ends of said guide means remote from said intersection point of said centrodes, and said intersection point are located at the corners of a notional equilateral triangle.

4. A knee orthosis according to claim 3, wherein the distance between said cam means corresponds to the side length of said notional equilateral triangle.

5. A knee orthosis according claim 1, wherein said intersection point of the centrodes associated with said guide means is located anteriorly, and the angle-bisecting line passing through said intersection point is inclined downwards in the direction from posterior to anterior between said guide means.

6. A knee orthosis according to claim 5, wherein said angle-bisecting line encloses with the longitudinal axis of the extended knee orthosis on the anterior side an angle selected from the group consisting of from 100° to 120°, and approximately 110°.

7. A knee orthosis according to claim 1, wherein said guide means extends over an arc angle of up to 60°.

8. A knee orthosis according to claim 1, wherein the position of said guide means and of said cam means associated therewith is such as to provide a hyperextension of up to −10°.

9. A knee orthosis according to claim 1, wherein the connecting line between said cam means in the extended state has an orientation selected from the group consisting of inclined upwards in the direction from posterior to anterior, inclined upwards in the direction from posterior to anterior at an angle of approximately from 5° to 20°, and inclined upwards in the direction from posterior to anterior at approximately 10°.

10. A knee orthosis according claim 1, wherein said guide means or continuous cam guide means form part of a first splint associated with a member selected from the group consisting of the femoral orthosis member, and the tibial orthosis member, and said cam means form part of a second splint associated with the respective other orthosis member.

11. A knee orthosis according to claim 1, wherein at least one joint is associated with stop means selected from the group consisting of posterior stop means, anterior stop means, posterior and anterior stop means, said stop means being operatively arranged to limit bending and/or extension.

12. A knee orthosis according to claim 1, wherein said joints are associated with at least one lateral outer covering by means selected from the group consisting of fastening means, a snap fastening, a hook fastening, and an interlocking fastening.
